(19) United States
(12) Patent Application Publication
(10) Pub. No.: US 2026/0198838 A1
(43) Pub. Date: Jul. 16, 2026

(54) SYSTEM FOR REHABILITATING FUNCTIONALITY AND RANGE OF MOVEMENT FOLLOWING ORTHOPEDIC, SPINE AND MUSCULOSKELETAL SURGERY

(71) Applicant: i-BrainTech Ltd, Tel Aviv-Yafo (IL)

(72) Inventors: Konstantin SONKIN, Tel-Aviv (IL); Brian FORSYTHE, Chicago, IL (US); Yoav ZAMIR, Tel Aviv (IL)

(73) Assignee: i-BrainTech Ltd, Tel Aviv-Yafo (IL)

(21) Appl. No.: 19/135,389

(22) PCT Filed: Dec. 7, 2023
(86) PCT No.: PCT/IL2023/051255

§ 371 Date: Jun. 4, 2025

Related U.S. Application Data
(60) Provisional application No. 63/431,084, filed on Dec. 8, 2022.

(51) Int. Cl. A61B 5/375; A61B 5/00; A61B 5/256; A61B 5/291; A61B 5/374; A61B 5/378

(52) U.S. Cl. CPC A61B 5/375; A61B 5/256; A61B 5/291; A61B 5/374; A61B 5/378; A61B 5/6803; A61B 5/7267; A61B 2505/09

(57) ABSTRACT

A system for testing and training a brain capability of planning and executing motion activity for a user who has sustained a functional movement deficit; said system comprising an electroencephalographic sensor arrangement attachable to the head of said user; a processor configured for receiving and analyzing electroencephalographic signals obtained from said user during visualization of an action; a memory storing instructions when executed by said processor for instructing said user to visualize executing said motion action; measuring electroencephalographic signals on said electroencephalographic sensor arrangement; calculating at least one characteristic selected from the following: a concentration index; a motor control index; an alertness index; providing said user with a feedback pattern based on at least one said concentration, motor control, alertness and motion readiness; recurring steps to be if needed.

### FIELD OF THE INVENTION

The present invention generally relates to systems and methods directed to rehabilitation of users with functional movement deficits, which are typically associated with orthopedic injury or surgery. The system may also be applied to users with general musculoskeletal, conditions. Additionally, the neurofeedback training movement performance system may be utilized post-operatively. Examples include orthopedic and spine trauma surgery, total hip, knee, ankle, shoulder, elbow, wrist and hand replacement (or transplantation) surgery, tendon transfer surgery, cervical spine disc replacement surgery, along with ligament, tendon, bone and cartilage reconstruction/realignment procedures of the spine, pelvis, hip, knee, foot and ankle, shoulder, elbow, hand and wrist. Other system applications include spine surgery users with degenerative and post-traumatic conditions.

### BACKGROUND OF THE INVENTION

The use of visualization to train motion capabilities for planning and executing motion activities.

Brain stimulation has been utilized to train the brain to enhance physical motion activities of the subject. It is demonstrated that by applying external electric current over specific brain regions, physical performance increases. This is well established with athletes. With application of brain stimulation training, performance improvement results are similar to those observed with organic, physiologic training. A disadvantage of brain stimulators is the duration of their effect. Upon removal, the training's impact lasts for approximately 20 minutes.

Commercially available technologies for neuro-interventions are devices for mental or cognitive state neurofeedback. These devices are simple in their application: sleek, low-profile electroencephalogram (EEG) sets that allow customers to train specific brain activation patterns, without the need for professional hardware or operator assistance. These products aim to target global and widespread brain processes such as relaxation, stress reduction and/or concentration. Using auditory or visual feedback, users and customers can learn how to induce a relaxed or focused mental state. These EEG devices focus on general mental processes as they have limited detection and recording capabilities.

Currently, these off-the-shelf EEG sets have a low signal to noise ratio, which limits the types of brain activities that they can detect and analyze. We expect this to evolve in the coming years, as technologies improve.

US 2019/0247662 discloses a method of facilitating a skill learning process or improving performance of a task, comprising: determining a brainwave pattern reflecting neuronal activity of a skilled subject while engaged in a respective skill or task; processing the determined brainwave pattern with at least one automated processor; and subjecting a subject training in the respective skill or task to brain entrainment by a stimulus selected from the group consisting of one or more of a sensory excitation, a peripheral excitation, a transcranial excitation, and a deep brain stimulation, dependent on the processed temporal pattern extracted from brainwaves reflecting neuronal activity of the skilled subject.

Impact of virtual embodiment and exercises on functional ability and range of motion in orthopedic rehabilitation (Matamala-Gomez et al. Nature Scientific Reports (2022) 12:5046): “Recent evidence supports the use of immersive virtual reality (immersive VR) as a means of applying visual feedback techniques in neurorehabilitation. In this study, we investigated the benefits of an embodiment-based immersive VR training program for orthopedic upper limb rehabilitation, with the aim of improving the motor functional ability of the arm and accelerating the rehabilitation process in users with a conservatively managed distal radius fracture. We designed a rehabilitation program based on developing ownership over a virtual arm and then exercising it in immersive VR. We carried out a between 3-group controlled trial with 54 users (mean age=61.80±14.18): 20 users were assigned to the experimental training group (immersive VR), 20 to the conventional digit mobilization (CDM) training control group, and 14 to a non-immersive (non-immersive VR) training control group. We found that functional recovery of the arm in the immersive VR group was correlated with the ownership and agency scores over the virtual arm. We also found larger range of joint movements and lower disability of the fractured arm compared with users in the Non-immersive VR and CDM groups. Feeling embodied in a virtual body can be used as a rehabilitation tool to speed up and improve motor functional recovery of a fractured arm after the immobilization period”.

To summarize, there is an established need to provide systems and methods for decoding and measuring brain patterns of users with functional movement deficits, who may have undergone orthopedic and musculoskeletal procedures, during the post injury or post-operative period to accelerate rehabilitation and to restore a healthy pattern of functional movements. Per above, some of the applications pertain to users who have undergone orthopedic and spine trauma surgery, total hip, knee, ankle, shoulder, elbow, wrist or hand replacement (or transplantation) surgery, tendon-transfer surgery, cervical spine disc replacement surgery, along with ligament, tendon, bone and cartilage restoration or reconstruction/realignment procedures of the spine, pelvis, hip, knee, foot and ankle, shoulder, elbow, hand and wrist. Other examples include spine surgery users with degenerative and post-traumatic conditions.

### SUMMARY OF THE INVENTION

It is hence one object of the invention to disclose a system for testing and training a brain capability of planning and executing functional motion activity for a user following orthopedic or musculoskeletal surgery procedures, as listed above.

It is an object of the present invention to provide a system for testing, training and preserving a brain capability of planning and executing functional motion activity for a user who has undergone orthopedic and musculoskeletal surgery; said system comprising:
a. an electroencephalographic sensor arrangement attachable to the head of said user;
b. a processor configured for receiving and analyzing electroencephalographic signals obtained from said user in response to said user visualizing a motion action, said motion action displayed visually to said user;
c. a memory storing instructions when executed by said processor for
   i. instructing said user to visualize executing said motion action;
   ii. measuring electroencephalographic signals on said electroencephalographic sensor arrangement;
   iii. calculating at least one characteristic selected from the following:
      1. a concentration index;
      2. a motor control index;
      3. an alertness index;
   iv. providing said user with a feedback pattern based on at least one said concentration, motor control, alertness and motion readiness;
   v. recurring steps c to e if needed.

It is a further object of the present invention to provide the aforementioned system comprising a display configured for providing a visual representation of said visualized motion action to said user.

It is a further object of the present invention to provide the aforementioned system wherein said displaying of said visual representation of saidvisualized motion action, measuring electroencephalographic signals on said electroencephalographic sensor arrangement and providing said feedback pattern are performed in a consecutive manner.

Another object of the invention is to disclose the system comprising a display configured for providing a visual stimulus to said user.

Another object of the invention is to disclose the system comprising a display configured for providing a visual representation of said visualized motion action to said user.

A further object of the invention is to disclose the user instructed to visualize executing said motion activity in response to displaying said visual stimulus.

A further object of the invention is to disclose the displaying said visual stimulus, measuring electroencephalographic signals on said electroencephalographic sensor arrangement and providing said feedback pattern performed in a consecutive manner.

A further object of the invention is to disclose the memory comprising an instruction of calculating said concentration index as a ratio of change of electroencephalographic signals at parietal-zone and frontal-zone electrodes at alfa-, beta- and theta-frequencies obtained from said electroencephalographic signals at parietal-zone and frontal-zone electrodes measured at rest.

A further object of the invention is to disclose the memory comprising an instruction of calculating said motor control index as a ratio of change of electroencephalographic signals at sensorimotor zone electrodes, at Mu-frequency obtained from said user in response to said visual stimulus below said electroencephalographic signals at sensorimotor zone electrodes measured at rest.

A further object of the invention is to disclose the memory comprising an instruction for calculating said alertness index as a ratio of change of electroencephalographic signals at parietal-zone electrode at alfa-frequency obtained from said user with open eyes over said electroencephalographic signals at parietal-zone with closed eyes.

A further object of the invention is to disclose the memory comprising an instruction of analyzing at least one of said concentration index, motor control index and alertness index of said user or a group of said users and presenting training progress data in a chronological manner.

A further object of the invention is to disclose the feedback pattern selected from the group consisting of a static avatar, a dynamic avatar, a text message, a sound pattern, a tactile pattern and any combination thereof.

A further object of the invention is to disclose the feedback pattern relating to a visual environment related to said motion action.

A further object of the invention is to disclose a method of testing and training a brain capability of planning and executing motion activity for users undergoing orthopedic and spine trauma surgery, total hip, knee, ankle, shoulder, elbow, and wrist replacement (or transplantation) surgery, tendon-transfer surgery, cervical disc replacement surgery, along with ligament, tendon, bone and cartilage restoration or reconstruction/realignment procedures of the spine, pelvis, hip, knee, foot and ankle, shoulder, elbow, hand and wrist. Other examples include spine surgery users with degenerative and post-traumatic conditions. The aforesaid method comprises steps of: (a) providing said system according to claim 1 for testing and training a brain capability of planning and executing motion activity for user undergone joint replacement; (b) instructing said user to visualize executing said motion action; (c) measuring electroencephalographic signals on said electroencephalographic electrode arrangement; (d) calculating said concentration index, motor control index and alertness index; (e) providing said user with a feedback pattern characterizing at least one of said concentration, motor control, alertness and motion readiness; and (f) recurring steps c to e if needed.

A further object of the invention is to establish HIPAA compliant digital platforms that will enable user subjects to compete and encourage one another to participate in brain training activities. All interactions amongst users would be through secure, HIPAA compliant portals, with users de-identified and restricted from contacting one another. users can monitor their progress via leaderboards and with comparisons to matched subjects, sharing similar demographics.

A further object of the present invention is to disclose a non-transitory computer-readable medium storing a set of instructions for testing and training a brain capability of planning and executing motion activity for a user who has undergone any orthopedic or musculoskeletal injury or surgery. The set of instructions comprising: one or more instructions that, when executed by one or more processors of a device, causes the device to: collect brain activity signals from defined brain zones while the motor visualization is executed by a user; analyze brain activity signals; form feedback characterizing the success of the user's motor visualization and; present visual feedback to a user. The feedback is dependent on the user's effort during the visualization. The feedback displays and scores increased excitability of predefined brain zones. These predefined brain zones can be selected from the group consisting of prefrontal cortex, motor cortex, corticospinal tract, and spinal motor neurons or any combination thereof.

A further object of the present invention discloses a non-transitory computer-readable medium storing a set of instructions for testing and training a brain capability of planning and executing motion activity for a user who has sustained or undergone any orthopedic or musculoskeletal injury or surgery. The set of instructions comprises: one or more instructions that, when executed by one or more processors of a device, causes the device to
a. instruct the user to visualize executing a motion action,
b. measure electroencephalographic signals on the electroencephalographic sensor arrangement
c. calculate at least one characteristic selected from the following
   i. a concentration index,
   ii. a motor control index,
   iii. an alertness index
d. provide the user with a feedback pattern based on at least one of concentration index, motor control index, alertness index and motion readiness;
   and; recur steps a to d if needed.

A further object of the present invention discloses the aforementioned non-transitory computer-readable medium with instructions for providing a display configured for providing a visual display of said motion activity to said user.

A further object of the present invention discloses the aforementioned non-transitory computer-readable medium having instructions enabling steps of game playing selected from the group consisting of point scoring, competition with others, rules of play, rewarding, ranking and any combination thereof

A further object of the present invention discloses the aforementioned non-transitory computer-readable medium having instructions enabling displaying of the motion activity, measuring electroencephalographic signals on the electroencephalographic sensor arrangement and providing the feedback pattern are performed in a consecutive manner.

A further object of the present invention discloses the aforementioned non-transitory computer-readable medium having instructions for calculating the concentration index comprises calculating a ratio of change of electroencephalographic signals at parietal-zone and frontal-zone electrodes at alfa-, beta- and theta-frequencies obtained from the user in response to the visual stimulus over the alfa-, beta- and theta-frequencies obtained from said electroencephalographic signals at parietal-zone and frontal-zone electrodes measured at rest.

A further object of the present invention discloses the aforementioned non-transitory computer-readable medium having instructions for calculating the motor control index comprises calculating a ratio of change of electroencephalographic signals at sensorimotor zone electrodes at Mu-frequency obtained from the user in response to said visual stimulus below said electroencephalographic signals at sensorimotor zone electrodes measured at rest.

A further object of the present invention discloses the aforementioned non-transitory computer-readable medium having instructions for calculating the alertness index comprises calculating a ratio feed of excess of electroencephalographic signals at parietal-zone electrode at alfa-frequency obtained from the user with open eyes over the electroencephalographic signals at parietal-zone with closed eyes.

A further object of the present invention discloses the aforementioned computer-readable medium having instrucitons for analyzing at least one of said concentration index, motor control index and alertness index of said user or a group of said users and presenting training progress data in a chronological manner.

A further object of the present invention discloses the aforementioned non-transitory computer-readable medium enabling a feedback pattern, the pattern may be selected from from the group consisting of a static avatar, a dynamic avatar, a text message, a sound pattern, a tactile pattern, and any combination thereof.

A further object of the present invention discloses the aforementioned non-transitory computer-readable medium having instructions for a feedback pattern, the pattern relating to a visual environment related to the motion action

A further object of the present invention discloses the aforementioned non-transitory computer-readable medium wherein said orthopedic surgery comprises artificial joint replacement.

A further object of the present invention discloses the aforementioned non-transitory computer-readable medium wherein said artificial joint replacement is selected from the group consisting of hip replacement, knee replacement, ankle replacement, shoulder replacement, elbow replacement, wrist/hand/finger joint replacement (or transplantation,) and cervical disc replacement.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 is a schematic diagram of a system for testing and training a brain capability of planning and executing motion activity;

FIG. 2a is a schematic view of an exemplary electroencephalographic cap;

FIG. 2b is a schematic view of an electroencephalographic sensor arrangement;

FIG. 2c is a schematic view of a signal amplifier;

FIG. 2d is front view of a processing unit;

FIG. 3 is a high level flowchart of aspects of the method for testing and training a brain capability of planning and executing motion activity

FIG. 4 is a flowchart of further aspects of the method for testing and training a brain capability of planning and executing motion activity

### DETAILED DESCRIPTION OF THE INVENTION

Rehabilitation following orthopedic and musculoskeletal surgery is typically a time consuming process, because long after the tissues have healed structurally and physiologically, much of the user's pre-injury brain capability is diminished.

The following description is provided, to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide systems and methods for testing, training, and preserving the brain capability for activating functionality and range of movement of a user who has undergone orthopedic or musculoskeletal surgery as described above. It will become apparent that the present invention herein described will substantially assist such a user and their caregivers in the course of rehabilitation to plan and execute functional physical actions. The trainee herein refers to an orthopedic or musculoskeletal surgery user who needs restoration of a healthy functional moving pattern following orthopedic or musculoskeletal surgery, as described above: users undergoing orthopedic and spine trauma surgery, total hip, knee, ankle, shoulder, elbow, and wrist replacement surgery, along with ligament, tendon, tendon-transfer, bone and cartilage restoration or reconstruction/realignment procedures of the spine, pelvis, hip, knee, foot and ankle, shoulder, elbow, hand and wrist. Other examples include spine surgery users with degenerative and post-traumatic conditions.

In the present disclosure, the term visualization and related terms are to be understood as equivalent to pragmatic mental representations of perceptual states that attribute action-properties. Visualizations and mental imagery attributes action-properties and can serve as the representational component of the immediate antecedent of the users actions.
The core of the present invention is a system and method which enables the following steps to be carried out: The user is instructed to visualize a motion activity, such as walking down stairs from a standing position, running, jogging, kicking, jumping, bending, flexing or any other like activity, involving, directly or indirectly, the anatomical structures that have undergone surgery. The visual and audio environment is immersive (stairs and surroundings) related to the motion action (walking down stairs) and can be displayed, such that the stairs and the user's avatar are observed on the system display as an immersive environment. This type of immersion can be termed tactical immersion or sensory motoric immersion and is experienced in an immersive environment when performing tactile operations that involve skill. Players feel “in the zone” while perfecting actions that result in success.

The electroencephalographic sensor arrangement, whilst the user is visualizing walking down the stairs in an immersive environment, is detected and analyzed, processed and displayed. In this way, the act of visualizing, (which is of course a brain activity), is displayed and the closeness to the ideal and desired motion parameters of walking and climbing can be assessed and represented. The user may then be automatically prompted to reiterate and improve the neurological activity of the brain to re-visualize and improve the appropriate brain activity associated with natural or normal walking

### Introduction to Free Moving, Articular Joints

Articular joints designed for free movement are called synovial joints, because they have capsules that contain synovial cells which generate a lubricant called synovial fluid, which reduces friction and facilitates smooth movement. There are six different types of synovial joints. They are classified based on the movements that they allow, like bending, straightening, rotating, pivoting, swiveling or gliding. The most familiar joints—and those most commonly replaced surgically—are ball and socket joints and hinge joints.

### Synovial Joints

Synovial joints are freely mobile (diarthroses) and are considered the main functional joints of the body. The joint cavity characterizes the synovial joint. The cavity is surrounded by the articular capsule, which is a fibrous connective tissue that is attached to each participating bone just beyond its articulating surface. The joint cavity contains synovial fluid, secreted by the synovial membrane (synovium), which lines the articular capsule. The articular surface is comprised of hyaline cartilage, which covers the articulating surface of each bone. The articular cartilage and the synovial membrane are continuous. Some synovial joints also have associated fibrocartilage, such as menisci, which assist with load transmission between articulating bones. Synovial joints are often further classified by the type of movements they permit. There are six such classifications: hinge (elbow), saddle (carpometacarpal joint), planar (acromioclavicular joint), pivot (atlantoaxial joint), condyloid (metacarpophalangeal joint), and ball and socket (hip joint). Ball and socket joints can move in multiple directions. Movement is possible because the ball-shaped end of one bone moves inside the cup-shaped end of another bone. The shoulder and hip are ball and socket joints.

Like the hinge on a door, hinge joints can close and open (bend and straighten), but only in one direction. The elbow and knee are hinge joints.

### Total Joint Replacement

Degenerative wear and tear, traumatic injury and inflammatory conditions like rheumatoid arthritis effect the body's joints, causing stiffness and pain that can make moving difficult. When symptoms become severe, one option to relieve pain and restore mobility is to surgically remove the damaged joint and to replace it with an artificial or prosthetic joint.

Regardless of its cause, joint pain and reduced mobility can significantly impact quality of life. Joint replacement may be recommended and is usually carried out by an Orthopaedic surgeon.

Hip replacement surgery and knee replacement surgery are most common, but replacement surgeries for other joints, including shoulder, elbow, hand and wrist, and foot and ankle replacement, are also possible. Depending on the joint being replaced, there are a number of different procedures. Artificial joints may be made from a variety of materials, such as Cobalt-Chrome, Titanium, Polyethylene, along with biologic joint resurfacing with autologous tissue and fresh or frozen allograft tissue. Recovering functional mobility, free of pain and discomfort following joint replacement or resurfacing surgery, is typically a long process lasting many months. It is the purpose of the present invention to harness the brain's ability to be neurologically trained through visual stimuli to accelerate the post operative functional rehabilitation process following orthopedic and neurosurgical procedures.

The systems and methods of the present invention will furnish physiotherapists, athletic trainers, sports performance specialists, biomechanists, physician assistants, nurse practitioners, physicians, surgeons and other caregivers, and users with relevant information for design of optimal training and rehabilitation protocols which will motivate users who have undergone orthopedic or musculoskeletal surgery to maintain a condition of high attentiveness. The present invention will allow for chronological data monitoring measuring the treatment effect. The information relating to the user's brain characteristics (e.g., concentration, motor cortex capacity traits) will assist caregivers in optimizing conditions for user execution and gamifying of tasks and exercises. Gamification has become increasingly important both in research and in practice. Particularly in long-term rehabilitation care processes, competitive and playful concepts are paramount to increasing motivation, compliance and adherence. This concept applies to most musculoskeletal and orthopedic disease conditions.

The present invention utilizes typical elements of gamification: point scoring, competition with others, rules of play, etc.

The present invention is configured to systematically record electroencephalographic signals relating to activation of particular brain regions related to movement such as the primary motor cortex. This is the reason for the use of high-quality EEG hardware-to ensure optimal data collection. By collecting and analyzing this unique data from the user, both online and offline, we create a rehabilitation environment that is highly specific and personalized which subsequently drives increased user performance.

Generally, the present invention system functions as a Brain-Computer Interface (BCI) for rehabilitation of users undergoing orthopedic and musculoskeletal procedures, as described above, and includes the following elements: (1) a Brain signal recording arrangement, (2) real-time signal analysis software, and (3) a user's front end (rehabilitation environment). Data is available on the “brain at work” (Parasuraman, R., and Rizzo, M. (2008). Neuroergonomics: The Brain at Work. New

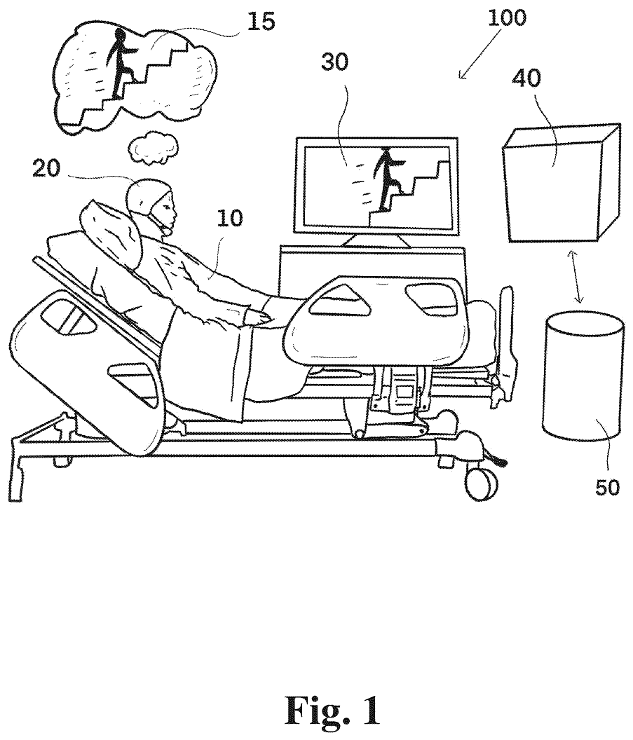

Reference is now made to FIG. 1 presenting a schematic diagram of system for testing and training a brain capability of planning and executing movements. Numeral 10 refers to a user to be tested. System 100 comprises memory unit 50 storing instructions for processing unit 40. The aforesaid processing unit is connected to electroencephalographic sensor arrangement 20 attachable to the head of said user such that electroencephalographic signals generated on the surface of the head of user 10 are detectable. user 10 is instructed to visualize executing a predetermined movement 15 in response to viewing a visual stimulus on display 30. Displaying the visual stimulus to user 10 is performed concurrently with measuring electroencephalographic signals on electroencephalographic sensor arrangement 20. According to the instructions stored in memory unit 50, the processing unit calculates a concentration index, a motor control index and an alertness index (described in detail below). A feedback message characterizing at least one said concentration, motor control, alertness and movement readiness are then provided to user 10. The feedback provided in the form of a static avatar, a dynamic avatar, a text message, a sound pattern, a tactile pattern is within the scope of the present invention.

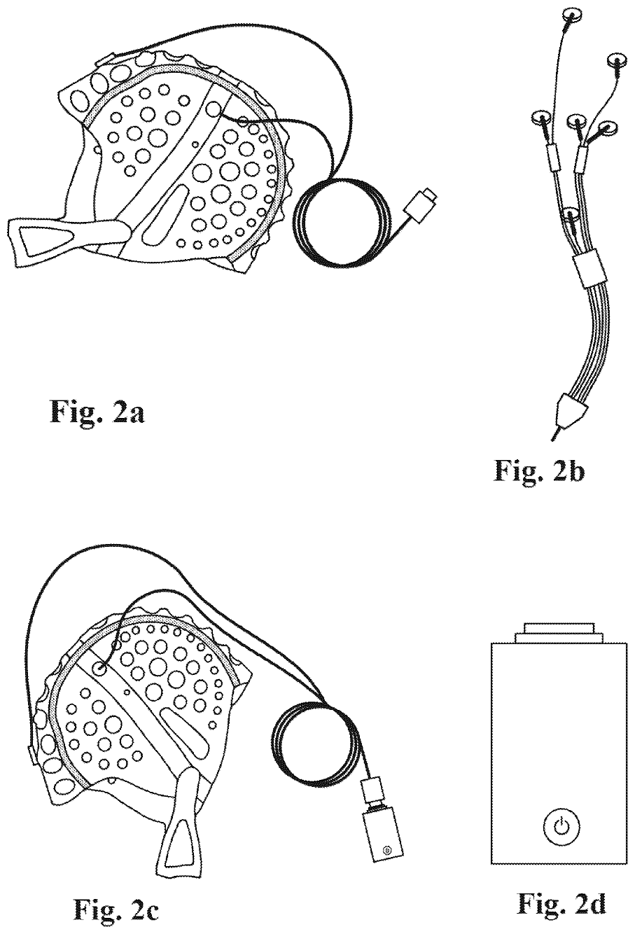

Referring to FIG. 2a, an exemplary electroencephalographic cap is made of an elastic synthetic fabric that comes in various sizes. The aforesaid cap positions the sensors exactly above the brain regions of interest. The standard EEG cap is known as the “10-20 system”. The “International 10-20 system” is a recognized method to describe the scalp electrodes' location. This standard testing system ensures a subject's study outcomes (clinical or research) could be compiled, reproduced, and effectively analyzed and compared employing the scientific method. The system is based on the relationship between the location of an electrode and the underlying area of the brain, specifically the cerebral cortex.

Referring to FIG. 2b, in the electroencephalographic sensor arrangement, each EEG sensor is recording the electric fields that are underneath it. The neurons communicate with one another with changes in electric charges. It creates a difference in the electric field around them. That is how we can decode the brain's functions—by analyzing these changes. The electroencephalographic sensor arrangement comprises sensors attachable at:
(1) frontal zone electrode, located on the midline of the frontal lobe
(2) parietal zone electrode, located on the midline of the parietal lobe;
(3-5) sensorimotor zone electrodes, attached to the motor cortex.

In addition, ground and reference sensors are utilized to collect signals.

The surface underneath the measurement sensors is cleaned prior to application, and it is brought into closer electrical contact with the skin surface by means of a conductive gel, to maximize signal-noise ratio, if necessary.

The sensors are connected to an amplifier (FIG. 2c) that increases the electric signal's magnitude. The amplifier is attached to the cap via a cord. It transmits the amplified electroencephalographic signals to a computer, smartphone, or tablet connected by a USB connector utilizing a wireless connection. Most of commercially available EEG signal amplifiers are usable with the present invention.

The EEG's data is streamed to the processing unit and structured with software according to a pre-set sensor montage. At the next step noise removal features are applied, by removing the electric network's static electric disturbances (, with a notch filter). The software, executed by a processing unit (FIG. 2d), allows the user to examine each sensor's connectivity level, which will cue he or she to add more gel if the impedance is too high or if a sensor malfunction is detected.

The EEG data is streamed per LSL protocol (Lab Streaming Layer,) and detected by the data-analyzing code.

Refernce is now made to the method for testing and training a brain capability of planning and executing movements. Method starts with providing system 100 for testing and rehabilitation of a brain capability for planning and executing movements described above. After instructing the user to visualize execution of the predetermined movement in response to displaying a visual stimulus, the aforesaid visual stimulus is displayed to the user to be tested concurrently with measurement of electroencephalographic signals on the electroencephalographic sensor arrangement. The electroencephalographic signals obtained are processed and the concentration index, motor control index and alertness index are calculated. Based on the calculated indexes, a feedback message characterizes at least one of concentration, motor control, alertness and movement readiness. The aforementioned steps are repeated as needed.

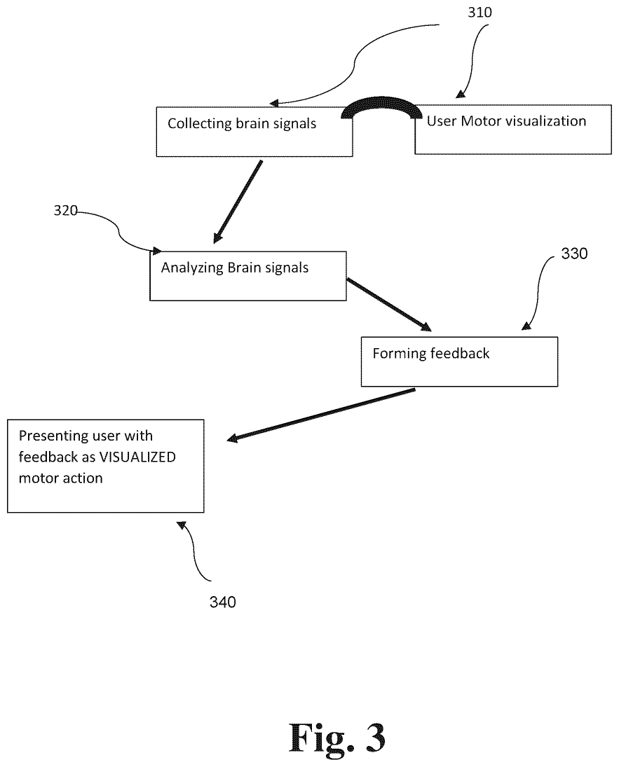

FIG. 3 shows high level aspects of the method: Collection of brain signals and motor visualization by the user are contemporaneous (310). Brain signals are analysed (320) and feedback is formed (330) which is presented to the user as visualized motor action (340)

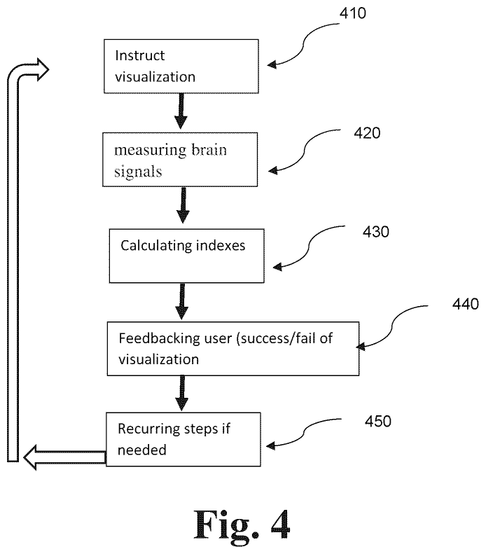

Reference is now made to FIG. 4 disclosing the method of the present invention comprising steps of:
providing the aforementioned system for testing and training a brain capability of planning and executing motion activity for user undergone joint surgery or joint replacement, or recovering from any orthopedic, musculoskeletal, or functional movement injury or disorder;
i. instructing said user to visualize executing said motion action (410);
measuring electroencephalographic signals on said electroencephalographic electrode arrangement (420);
iii. calculating said concentration index, motor control index and alertness index (430);
iv. providing said user with a feedback pattern characterizing at least one of said concentration, motor control, alertness and motion readiness(440);
v. and recurring steps iii to v if needed (450).

Further details on the method and system:
EEG data is received over an LSL socket. The data analysis includes filtration. The EEG raw-data is analyzed in time windows with a shift (e.g., 500 or 1,000 samples with 50% shift) in each cycle. The goal of the analysis is to extract relevant brain function features that contribute to successful performance of motion control.

The level of a person's concentration and activity level in the motor cortex are deducted from fluctuations of the power of certain frequency bands. We detect these with at least five scalp electrodes.

Calculation of the “concentration” (or-brain engagement) index is performed according to the following algorithm:
Raw data is first filtered using an IIR filter, with half-power frequencies for a frequency range [alpha: 8 to 11 Hz; beta: 16-22Hz; and theta 4-7Hz] on the data from sensors attached to parietal zone (alpha) and frontal zone (beta and theta). The power of each frequency band (alpha, beta, and theta) is calculated with a “bandpower” method based on the relevant, filtered data. The concentration index for each cycle is the ratio of the powers of beta, theta and alpha.

Threshold—accuracy: At the beginning of each session, the system will determine a baseline that characterizes each user. The user will sit still in front of an instructed simulation for approximately several minutes (2 minutes by default) to create an open-eyes baseline. The system collects the indices at baseline and and sets a user threshold. This sets the user's customized boundary. If exceeded, the system determines that the user's concentration is sufficiently high enough to provide positive feedback.

According to an exemplary embodiment of the present invention, the aforesaid threshold can be set as a sum of values of lower bound and a compound of difficulty level and the difference between upper and lower bounds.

Upper bound is the average of the indices, Lower bound is the average minus two standard deviations of the index, and the Difficulty level is a value set by the user to adjust the challenge level to the user's abilities.

Training utilizing in-game feedback: During the game, the indices are continuously calculated and compared with the user's threshold baseline through the backend module. Suppose the concentration index's current value exceeds the threshold set for it. In that case, the module will send the game simulation a message) to grant positive user feedback. As an example, in a stepping game, the user will notice it through shrinkage of a circle indicating the target on the floor, making the step more accurate. After several attempts during which the user exceeded the threshold in the current trial (for example, in a stepping game, at least five times within 8 seconds), intermittent feedback is given (in the stepping game,—the walk or gait of the user would be safe, sure, and steady).

The algorithm for calculation of the motor control index is the following:
First, raw data is filtered using an IIR filter, with half-power frequencies for a frequency range [Mu: 12 Hz to 15Hz] for sensorimotor zone sensors, such as C3, Cz and C4 above the motor cortex. The bandpower function calculates the power of the Mu frequency band and the filtered data from these channels. The index from sensorimotor zone sensors, such as C3 and C4 is later used to evaluate current Mu desynchronization which is an action preceding movement.

Threshold—Kick power: all Mu power indices calculated during the acquisition of open-eyes baseline data from the sensorimotor zone, such as C3 and C4 electrodes are utilized to evaluate the user pattern of Mu rhythm of the specific user.

For example, the average Mu power of locations C3 and C4 (above the left motor cortex that controls the right-side limbs and vice versa) is utilized as the motor brain activity threshold.

Training based on in-game feedback: during the game, the current Mu power is continuously compared with the average value of the data collected in the open-eyes baseline. Specifically, the feedback is defined as positive if the sum of momentary Mu power and compound of difficulty lever and STD of MU at baseline is smaller than the mean MU at baseline.

“Mu power” represents the momentary Mu value; Difficulty level is a value set by the user to adjust the challenge level to the user's abilities; STD of Mu at baseline is the standard deviation of all Mu indices collected at open-eyes baseline. Suppose the current Mu power plus a portion of the standard deviation is lower than the average Mu at baseline. In that case, there is an activation of the motor cortex—and positive feedback is given.

The algorithm of calculation of the alertness (sleep) index is the following:
The power of the alpha band in the central parietal sensor, such as Pz, is collected at closed-eyes baseline (several minutes, 2 minutes by default), immediately after open-eyes baseline collection,) and compared to the alpha power open eyes baseline. Alpha power at this location is known as relating to the user's level of arousal; higher alpha power is usually associated with tiredness.

Threshold—Sleep detection: A moving average for both open-eyes and closed-eyes baseline data is calculated and compared. First, it is based on averaging of one time-window, then the interval increases until the moving average of closed eyes is at least 2 standard deviations higher than the alpha values at open-eyes state. The moving average with the minimal time interval size that satisfies this condition is set as the threshold for “sleep detection”.

A walking game is an initial example—the same concept is applied to different gaming and training environments.

Applying data from early adopters in the field, advanced ML tools can be utilized, such as clustering algorithms, SVM classifiers, and Artificial Neural Networks to create a powerful pattern detection mechanism that is highly user-specific, robust to EEG noise and provides users with rich data-driven insights.

The final element of the system that the present invention addresses is the training environment for the user. The interface exposes the user to a real-time representation of his or her brain activity. With respect to brain data acquired with the EEG, it is essential to close the loop in a neurofeedback learning process. We designed the products to allow users to train brain functions that are crucial to improving their performance. Thus far, we have presented the underlying brain processes that our system tracks-motor imagery under high concentration levels. These brain processes are shared between different kinds of movements. However, current research indicates that a feedback environment for this type of neural training is much more efficient in a proper learning environment. Therefore, for each type of movement, unique virtual environments should be developed. The goal is to allow people to train in a familiar environment, which will enable them to efficiently transfer their learning into the ‘real-world’.

For example, the user is instructed to sit still, focus on the monitor, and visualize that he or she is walking towards a target set on the screen. For each attempt by the user, the system allows a time period of 8 seconds. The neural activity in the case of real and visualized ‘movement” is very similar. If the system detects strong activation of specific bands in specific brain areas related to the movement, an indicator of movement power will increase. This correlation between neural activation and a symbol presented on the screen allows the user to gain intuitive control of the activity of this neural network that is most important in controlling their leg during walking.

With practice, the user learns how to control these brain areas, but he or she is also inducing changes in the way they are ‘wired’. The brain is an organ that changes continuously: neuronal connectivity changes and evolves, and networks of neurons are created or enhanced with repeated activation. These changes are the building blocks of learning, and they underlie the reason we improve with training and repetition. This is facilitated by the immersive nature of the system and method.

The present invention thus provides a system and method for testing and training a brain capability of planning and executing motion activity, following orthopedic or musculoskeletal injury or surgery. It should be emphasized that the disclosed invention is usable in any kind of human motion activity where a user's joint has been repaired, reconstructed, resurfaced, transplanted, tendon-transferred or replaced for whatever reason. users with any functional movement deficit may also benefit from said invention. Etiology for said conditions include traumatic and degenerative pathologies.

1-42. (canceled)

43. A system for rehabilitation of a user post orthopedic surgery and/or musculoskeletal injury, said system comprising:
an electroencephalographic sensor arrangement attachable to the head of said user;
a processor configured for receiving and analyzing electroencephalographic signals obtained from said user;
iii. a memory storing instructions for execution by said processor, comprising instructions for:
a. instructing said user to visualize executing a motion action;
b. measuring electroencephalographic signals on said electroencephalographic sensor arrangement;
c. calculating at least one characteristic selected from the following: a concentration index; a motor control index; an alertness index;
d. providing said user with a feedback pattern based on at least one of the following indicators: an accuracy of the motion action based on said concentration index, a power of the motion action based on said motor control index, and motion readiness based on said alertness index;
e. iterating steps b to d; and
f. generating a dynamic visual representation of the motion action being executed by a character in a virtual environment according to at least one of the power, accuracy, and motion readiness obtained during the iterations.

44. The system according to claim 43, wherein said memory comprises an instruction of calculating said concentration index as a ratio of change of electroencephalographic signals at parietal-zone and frontal-zone electrodes at alpha-, beta- and theta-frequencies obtained from said electroencephalographic signals at parietal-zone and frontal-zone electrodes measured at rest.

45. The system according to claim 43, wherein said memory comprises an instruction of calculating said motor control index as a ratio of change of electroencephalographic signals at sensorimotor zone electrodes, at Mu-frequency obtained from said user in response to a visual stimulus presented on a display below said electroencephalographic signals at sensorimotor zone electrodes measured at rest.

46. The system according to claim 43, wherein said memory comprises an instruction for calculating said alertness index as a ratio of change of electroencephalographic signals at parietal-zone electrode at alpha-frequency obtained from said user with open eyes over said electroencephalographic signals at parietal-zone with closed eyes.

47. The system of claim 43, wherein said memory includes instructions for feeding said electroencephalographic signals into a machine learning model, and obtaining detected patterns robust to EEG noise from the machine learning model, wherein at least one of the concentration index, the motor control index, and the alertness index, is computed according to the detected patterns robust to EEG noise.

48. The system of claim 43, wherein said memory includes instructions for monitoring connectivity level of each sensor of the electroencephalographic sensor arrangement, and generating an indication for adding more gel.

49. The system of claim 43, wherein said memory includes instructions for:
computing a baseline threshold based on at least one of the concentration index, the motor control index, and the alertness index calculated during a baseline collection time interval,
wherein the feedback pattern is generated according to at least one of the concentration index, the motor control index, and the alertness index calculated during the iterations relative to the baseline threshold.

50. The system of claim 43, wherein said memory includes instructions for dynamically adapting the feedback pattern to adjust a difficulty and/or challenge level according to a user's abilities indicated by at least one of the calculated concentration index, the motor control index, and the alertness index.

51. The system according to claim 43, wherein said orthopedic surgery comprises artificial joint replacement, selected from the group comprising: hip replacement, knee replacement, ankle replacement, shoulder replacement, elbow replacement, wrist/hand/finger joint replacement, and cervical disc replacement.

52. A method of treating a user post orthopedic surgery and/or musculoskeletal injury, said method comprising:
i. instructing said user to visualize executing a motion action;
ii. measuring electroencephalographic signals by a electroencephalographic sensor arrangement during the visualization of the execution of the motion action;
iii. calculating at least one characteristic selected from: a concentration index, a motor control index and an alertness index;
iv. providing said user with a feedback pattern based on at least one of the following indicators: an accuracy of the motion action based on said concentration index, a power of the motion action based on said motor control index, and motion readiness based on said alertness index;
v. iterating steps ii to iv; and
vi. generating a dynamic visual representation of the motion action being executed by a character in a virtual environment according to at least one of the power, accuracy, and motion readiness obtained during the iterations.

53. The method according to claim 52, wherein said step of calculating said concentration index comprises calculating a ratio of change of electroencephalographic signals at parietal-zone and frontal-zone electrodes at alpha-, beta- and theta-frequencies obtained from said user in response to a visual stimulus presented on a display over said alpha-, beta- and theta-frequencies obtained from said electroencephalographic signals at parietal-zone and frontal-zone electrodes measured at rest.

54. The method according to claim 52, wherein said step of calculating said motor control index comprises calculating a ratio of change of electroencephalographic signals sensed by electrodes positioned at a sensorimotor zone and at Mu-frequency, while a visual stimulus is presented on a display, relative to electroencephalographic signals measured at rest.

55. The method according to claim 52, wherein said step of calculating said alertness index comprises calculating a ratio of excess of electroencephalographic signals at parietal-zone electrode at-frequency obtained from said user with open eyes over said electroencephalographic signals at parietal-zone with closed eyes.

56. The method according to claim 52, comprising steps of analyzing at least one of said concentration index, motor control index and alertness index of said user or a group of said users and presenting training progress data in a chronological manner.

57. The method according to claim 52, wherein said orthopedic surgery comprises artificial joint replacement selected from a group comprising: hip replacement, knee replacement, ankle replacement, shoulder replacement, elbow replacement, wrist/hand/finger joint replacement, and cervical disc replacement.

58. The method according to claim 52, wherein the orthopedic and/or musculoskeletal injury and/or surgery is selected from: spine trauma surgery, spine surgery on a subject with degenerative and/or post-traumatic condition, joint transplant surgery, ligament and/or tendon surgery, tendon-transfer surgery, cervical spine disc replacement surgery, bone and/or cartilage restoration or reconstruction/realignment procedures of the at least one of: spine, pelvis, hip, knee, foot, ankle, shoulder, elbow, hand, and wrist.

59. The method according to claim 52, wherein the surgery is selected from: repaired, reconstructed, resurfaced, transplanted, tendon-transferred or replaced.

60. The method according to claim 52, further comprising diagnosing the user with a functional movement deficit secondary to the orthopedic and/or musculoskeletal injury and/or surgery, wherein the subject is treated for rehabilitation of the functional movement deficit.

61. The method according to claim 52, wherein the motion action that the user is instructed to visualize is selected from physical therapy activities including: walking down stairs from a standing position, running, jogging, kicking, jumping, bending, and/or flexing.

62. The method according to claim 52, wherein the motion action that the user is instructed to visualize comprises an activity, involving, directly or indirectly, at least one anatomical structure that have undergone surgery and/or have been injured.

63. The method according to claim 52, wherein the user is instructed to sit or stand in front of a display and visualize movements and/or physical therapy exercises prompted on the display, wherein a quality of the visualization is continually presented on the display for providing objective data-driven feedback and promoting increase in the target brain activity.

64. The method according to claim 52, wherein the motion action that the user is instructed to visualize comprises sitting still, focusing on a display, and visualizing walking towards a target presented on the display.
